(19) United States
(12) Patent Application Publication
(10) Pub. No.: US 2007/0232971 A1
(43) Pub. Date: Oct. 4, 2007

(54) Padded orthopaedic splint device

(76) Inventor: John Louis Stanton, Clarksville, TN (US)

Correspondence Address: David D. Winters, 2277-C, Suite 237, Wilma Rudolph Blvd., Clarksville, TN, 37040-5898, US

(21) Appl. No.: 11/393,590

(22) Filed: Mar. 30, 2006

(51) Int. Cl. A61F 5/00

(52) U.S. Cl. 602/6

(57) ABSTRACT

Padded orthopaedic splint device with splint core of flexible fabric that may be cured to hardness, including a sleeve surrounding the splint core with polyester scrim in net-like or web-like configuration coated with polyvinylchloride.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

Lexicon

Use of the word “impervious” in this patent is intended to mean “not affected by.”

For the purposes of this patent application, common and predominant professional medical usage governs, defining a “splint” as a support device which is rigid and immobilizes, but does not encase, the member supported. This is in differentiation from a “cast” is defined as a “rigid encasement of a member to immobilize that member.”

### BACKGROUND OF THE INVENTION

This invention relates generally to the field of orthotic, or orthopedic devices and more specifically to a device and process for lining and/or cushioning orthopedic splints, particularly, an improved medical splint formed of a moisture curable resin and a water tolerant, water permeable padding, which either encases the splint and/or provides a soft surface for contact with the patient.

Various orthopedic devices and processes are commonly used to reinforce or immobilize one or more portions of the human body, usually a limb or joint. Splints and casts being the most common means, they are definitively different in concept and execution. Splints are frequently removable, and may even be worn for only a short period each day, or only when needed to alleviate some painful or injurious symptoms.

In general, splints do not completely encase the limb or body part being protected. They are applied in strips to one, two, or three sides of the limb, but do not circumferentially enclose the limb. This is an important attribute of the splint because it allows for expansion or contraction of the splint around the body part, which allows for swelling as it increases or decreases during the early phases of healing. Thus, the splint is often used early in the care of a fracture and then later a cast is substituted. In some cases, of course, the splint is used until the fracture heals, performing long-term immobilization.

Early splints were commonly made from layers of plaster-impregnated fabric. These plaster and fabric layers were dipped in water. Then, excess water was removed and the splints were placed on cotton layers and applied to the limb to be immobilized. This was in turn held in place with some type of elastic bandage.

These splints were padded with cotton at first, and later, with synthetic material. Splints are known to frequently migrate from their desired positions during activity. This migration frequently results in skin irritation and laceration. Perspiration coupled with a lack of circulation further allows irritation and maceration of the skin under the splint, which may result in the growth of bacteria and skin erosion. If untreated, this condition can lead to infection.

Subsequently, splints were developed that are made from a flexible fabric impregnated with a moisture curable resin. The user encases such a splint in synthetic padding. Other embodiments include splints made with a layer of synthetic material on one side to pad between the patient and the brace, juxtaposed to a water impervious outer layer, typically of polypropylene, which limits air circulation to cover the outside surface of the brace.

They require, therefore, forms and qualities of padding different from those of a cast.

Fiberglass resin-containing splints have a very high strength to weight ratio, are very light, and can be made relatively porous, to permit a flow of air through the splinting material. Examples of splinting materials as described above are seen in U.S. Pat. No. 5,171,208 (Edenbaum, et al), U.S. Pat. No. 5,318,504 (Edenbaum, et al), U.S. Pat. No. 5,520,621 (Edenbaum, et al), and U.S. Pat. No. 6,780,162 (Pounder, et al).

These moisture curable brace materials are sensitive to the presence of even minute amounts of moisture. To guard against this moisture, the brace material may either be packaged in units of a wide variety of different shapes and sizes to fit various needs, or encased in some type of packaging that can be cut and resealed to prevent moisture entry.

To employ a moisture curable brace, the splint material must first be immersed in water to activate it. Then it is wrung out and applied. This results in a wet layer of material in direct contact with the patient's skin, which may be uncomfortable and irritating and can, in time, cause maceration and sloughing of the skin. Even devices that have a moisture permeable layer between synthetic layers of padding tend to accumulate moisture within the padding against the skin.

Such splints that have a thick synthetic layer next to the skin or that have a layer of water impermeable polypropylene, often cause retention of moisture such as perspiration that would otherwise evaporate. The moisture builds up under the impermeable layer and may cause odor and/or tissue damage.

Other devices have been described, for example in U.S. Pat. No. 4,770,299 (Parker), U.S. Pat. No. 4,869,046 (Parker), and U.S. Pat. No. 4,899,738 (Parker), which use hydrophobic material on the patient contact side, while still requiring water immersion of the brace material. The required immersion and “squeezing out” of the in-place material results in water being trapped or retained in the spaces of the brace. This then presents a wet surface to the patient and may trap moisture next to the patient's skin.

After the water activated splint is applied, the patients will often have water running from the ends of the splint, dripping on clothes, patient, floor, furniture, and the like. Such a splint is unaffected by water. However, while a fiberglass splint may be tolerant of water, a cotton and/or synthetic liner used with it is not. Such a liner holds water, dries slowly, and retains moisture against the skin. It is also weakened and tends to clump and bunch up when wetted. The accompanying lack of air to dry the skin could also encourage maceration under the splint, opening it to attack by bacteria. Such skin erosion and subsequently formed pustules can cause extreme discomfort. Furthermore, unpleasant odor may develop due to retention of body oils and perspiration in the padding. An ability to immerse the splinted member without damage could be valuable in helping to prevent this.

Other patents disclose splints that are prepackaged in convenient lengths. Still others describe splint material that is in a continuous strip and must be cut to length. In both cases, the resulting splint often needs to be revised and trimmed to appropriately fit the extremity. Further, the synthetic material commonly found in splints has a disadvantage of being difficult to cut since it needs to be rather thick and requires considerable effort to penetrate.

Other splint designs have a smooth outer surface that may fail to hold the required elastic bandage wrap in its proper place. The bandage may slide up and down on the smooth outer surface.

The following other patents and product literature are representative of the previously known art in the field or associated fields.

U.S. Pat. No. 4,194,041, (Gore, et al) describes a waterproof and breathable polytetrafluoroethylene (hereinafter PTFE) laminate which allows high water vapor transmission even under adverse climatic conditions. Such laminates in modified forms are commercially available from W. L. Gore & Associates. The waterproof and breathable laminate described in this patent consists of a flexible outer layer of micro porous hydrophobic material, such as porous, expanded PTFE, (hereinafter ePTFE) and a second interior layer of continuous hydrophilic elastomeric material attached to the interface of the outer layer. The interior layer does not permit passage of liquid water. The material is specified for use in the fabrication of waterproof, breathable garments, tents, and the like. It is not specified or suggested for orthotic use.

U.S. Pat. No. 4,989,593 (Campagna, et al) discloses a rigid orthopedic splint which includes padding treated with a fluoro-chemical or silicone. The padding is able to shed water, rapidly promoting comfort for the user.

U.S. Pat. No. 5,016,622, (Norvell)-discloses an orthopedic cast and padding assembly, but not a spint device, having a water impermeable, water vapor permeable membrane in contact with the skin, a resilient padding layer of conventional material, and a plaster or resin/glass fiber outer immobilizing layer. The assembly is said to increase the comfort of a wearer and reduce bacteria contained within the cast.

Another cast material is found in U.S. Pat. No. 5,102,711 (Keller, et al). This patent discloses a flexible, breathable, composite having a middle layer of conventional man-made or natural fiber padding and a top and bottom layer comprising a sheet of water impermeable, water vapor permeable film bonded to the middle layer. Preferably the top and bottom layers comprising: (a) a flexible first sheet of hydrophobic material and (b) a continuous hydrophilic sheet attached to or penetrating the first sheet forming a barrier to passage of fluids. This composite material is useful in applications such as liners under orthopedic casts and as thermal insulation in apparel.

U.S. Pat. No. 5,397,628, (Crawley, et al)-discloses a body protection material having an inner layer of ePTFE laminated to an outer layer of substantially air impermeable cellular rubber.

U.S. Pat. No. 5,916,184 by McKeel teaches a polyethylene cast liner composed of closed-cell thermal foam polyethylene. The material is configured in sheets with apertures penetrating from one side to the other, these apertures being circular in form and aligned in rows. Only a relatively small percentage of the padding material surface area is occupied by these voids, which exist primarily for drainage and ventilation purposes.

In contrast to this, the instant technology comprises not a sheet, but a web configuration, whereby a checker-board form is created, having alternating pillows and spaces. This form allows for improved stretching and shaping and allows the padding to better conform to the shape of the underlying body part, while also providing improved ventilation and drainage. Additionally, since the instant art comprises polyvinylchloride (PVC), it displays improved nonhydrophilic qualities and avoids the common problem of allergies and sensitivities frequently resulting from exposure to polyethylene and latex based substances.

Previously taught technologies do not overcome the problem of retained dampness once liquid gets inside the lining and reaches the user's skin. None of the protective liner systems do, nor should, provide a water tight seal against immersion. Additionally, in conditions of immersion, the previously taught and otherwise valuable quality of being water impermeable while water vapor permeable becomes moot because liquid water will inevitably enter the interior of the liner by bypassing the edges of the protective materials. The problem to be addressed then becomes one of quickly getting the skin dry again. Without the ability to efficiently dry the padding under splints, immersing, or otherwise wetting them becomes unfeasible.

In summary, use of splints often causes discomfort to the wearer and may cause injury. To overcome this, there exists a need, unfilled by the above-taught arts, for, effective, inexpensive, fast drying, non-allergenic, waterproof splint padding that can be used in direct contact with the skin of a wearer, and a method for using it. Previous splint liner applications are exemplary of the need for waterproof fabrics and of the lack of suitable fabrics in the previous technologies to fulfill these needs.

### BRIEF SUMMARY OF THE INVENTION

The method presented in this patent has been exercised on volunteer human subjects with unqualified success. Properly administered, it provides for significantly improved comfort, freedom of activities and freedom from inflamation or infection over previously known technologies.

In its preferred embodiment as presented herein, the invention comprises a splint core of flexible fabric that may be cured to hardness and a sleeve surrounding the core, comprising polyester scrim in net or web like form coated with polyvinylchloride. It also includes a dispenser that can be repeatedly opened and resealed to an air and water tight condition.

The present invention is, thus, a unitary splinting device that includes a water permeable, water tolerant, hydrophilic padding. This padding is comprised of a polyester scrim with alternating patches of dense weave between the warp and the weft. This results in a checkerboard pattern, which when dipped in polyvinylchloride foam, produces a soft, water tolerant padding that has alternating open and closed patches to allow the passage of air and moisture. This padding, in one embodiment, is used to encase a resin-impregnated moisture curable material.

The device also includes the means for holding the layers together to form the unitary device. In other embodiments, the device includes water tolerant, water permeable padding for contact with the patient, under a liquid activated splinting brace layer, both of these under a non-hydrophilic, water permeable, second, outer protective layer outside the splinting layer. The device may also include the means for holding the layers together in a unitary manner and the outer layer contains or is covered at least in part with a hook portion of hook-and-pile/fabric hook liner to prevent slippage of the elastic bandage holding it against the patient. Finally, the system may package such a splinting product within a moisture impervious dispenser with means for resealing the dispenser after materials are dispensed.

This polyvinylchloride padding, which either encases the splint or lies against one surface of the splinting material, has the advantage of being light-weight, easy to cut, water and moisture tolerant, and moisture permeable. It allows easy wetting of the resin-hardening material encased within by simply placing it directly under water. Furthermore, because of the soft nature of this padding, the splint can be easily wrung dry, and does not require that the brace material be dried separately prior to applying it to the patient.

The preformed splint is used for immobilization of an injured limb or joint. The body part in question is measured and the splint is cut to the appropriate length to fit the extremity. Either the splint is immersed under water or water is run over the splint. The water penetrates quickly to the fiberglass core through the open weave of the PVC pattern covering. Once the fiberglass core is wet, the splint material is rolled up and squeezed to eliminate excess water and it is then laid out and if necessary dried with a towel. The splint is applied to the limb in question and then held with an elastic bandage. The splint is then allowed several minutes to harden.

An air impervious sheath to contain the splint material and limit air contact is also disclosed. In one embodiment, the splint and the encasing sheath is placed in a collapsible pouch with a Ziploc™ type fastener across the top. In a second embodiment, the cut end of the sheath is folded over and a clip is used to maintain a moisture impermeable seal on the sheath itself. After the splint is cut and replaced, excess air is expelled and the Ziploc fastener is closed. In another alternative embodiment, the sheath is folded and placed in a collapsible pouch with a rigid top. The rigid top has an opening sufficient to allow the splint material to be removed. This rigid opening can then be closed, after expelling excess air, with a cover or plug to prevent air and moisture from passing inside. Other means of hermetically sealed containment may be substituted.

### OBJECTS OF THE INVENTION

The primary object of the invention is to provide a unitary splint and padding device with both brace and padding organic to the splint device.

Another object of the invention is to provide unitary splint and padding material that can be easily cut and fashioned to fit the patient

Another object of the invention is to provide a unitary splint and material that can be easily moistened without running water into the ends of the device or requiring that the device be totally immersed.

Another object of the invention is to provide splint-padding material that does not slip out of position on the patient and that does not allow slippage of an elastic bandage used to hold it in place.

Another object of the invention is to provide a dry patient contact layer for a splint system that will allow water vapor and liquid passage.

Another object of the invention is to provide a unitary splint and padding device that can be repeatedly wetted and dried without adversely effecting its qualities of padding and support.

Another object of the invention is to provide a splint with soft, resilient, padding, organic to it.

Another object of the invention is to provide a splint with water tolerant padding.

A further object of the invention is to provide a splint with tear resistant padding.

Yet another object of the invention is to provide a splint with padding that is essentially inert or not chemically reactive.

Still yet, another object of the invention is to provide a splint padding that is hypo-latex allergenic.

Another object of the invention is to provide a splint padding that has a high coefficient of friction.

Another object of the invention is to provide a splint padding material that is re-usable.

A further object of the invention is to provide a splint-padding sleeve that is inexpensive.

A further object is to provide a container from which unitary splint and padding devices may be repeatedly dispensed in varying quantities of choice, without degrading the quality of the remaining splint and padding material still incased in the container.

It is an objective for this invention to provide a unitary splinting device that represents a relatively dry surface to the patient when applied.

Other objects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1. Front view of the padding with a portion of the PVC foam removed to reveal the polyester scrim.

FIG. 2. Cross section view of the padding shown in FIG. 1.

FIG. 3. Front view of a portion of a fiberglass splint.

FIG. 4. Cross section of splint shown in FIG. 3.

FIG. 5. Front view of an alternative embodiment of a splint.

FIG. 6. Cross section of the splint shown in FIG. 5.

FIG. 7. Front view of an alternative embodiment of a splint.

FIG. 8. Cross section view of the splint shown in FIG. 7.

FIG. 9. Cross section view of a splint with storage system.

FIG. 10. Cross section view of an alternative storage system for the splint.

### DETAILED DESCRIPTION—LIST OF COMPONENTS

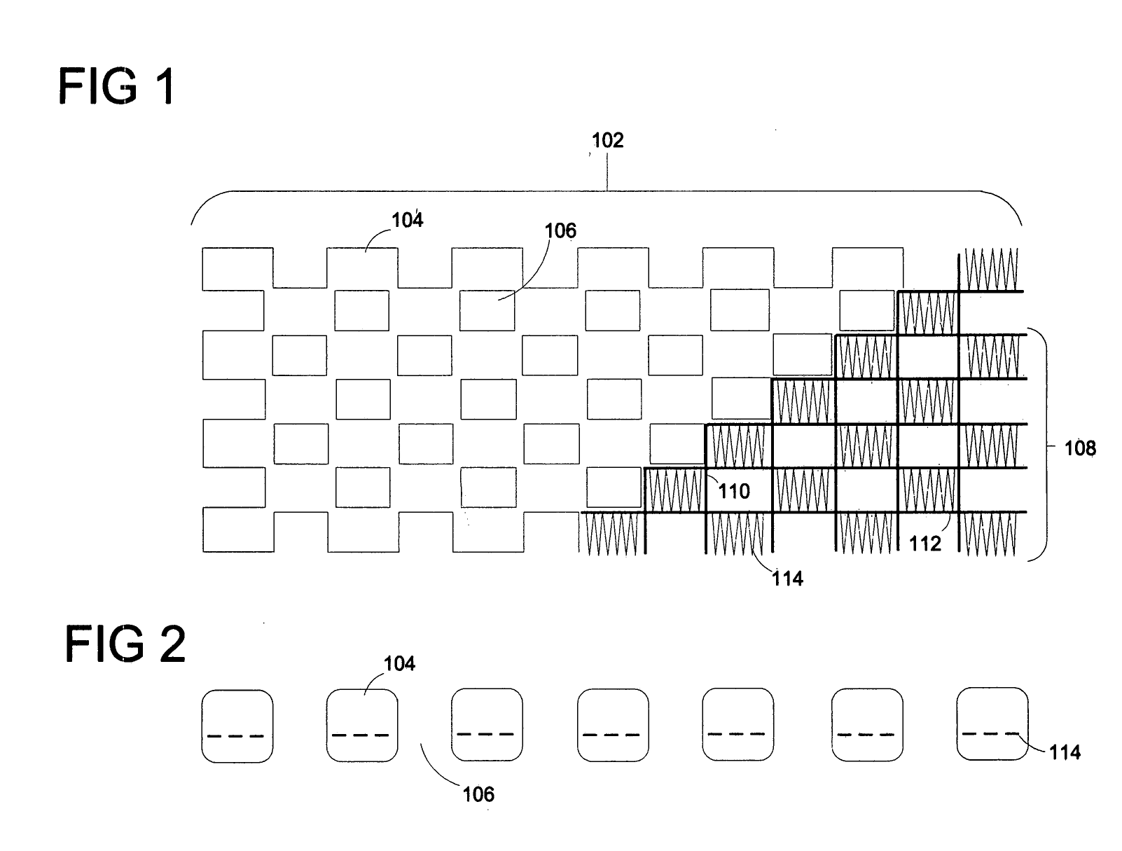

FIG. 1:
104. Pillows
106. Spaces
108. Scrim
102. Padding
110. Warp
112. Weft
114. Dense weave

FIG. 2:
102 Padding
104 Pillows
106. Spaces
108. Scrim
114. Dense weave

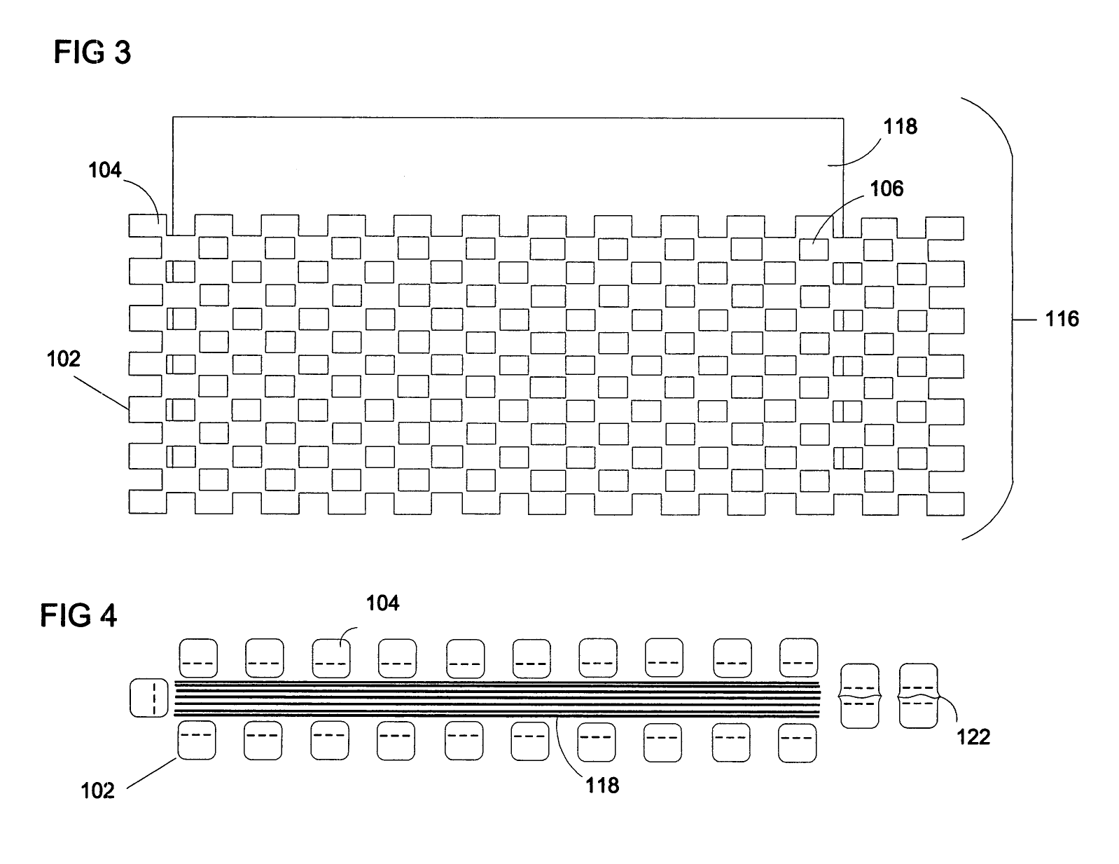

FIG. 3:
102. Padding
116. Unitary splint device
118. Resin-impregnated core

FIG. 4:
102. Padding
118. Resin-impregnated core
122. Heat seal

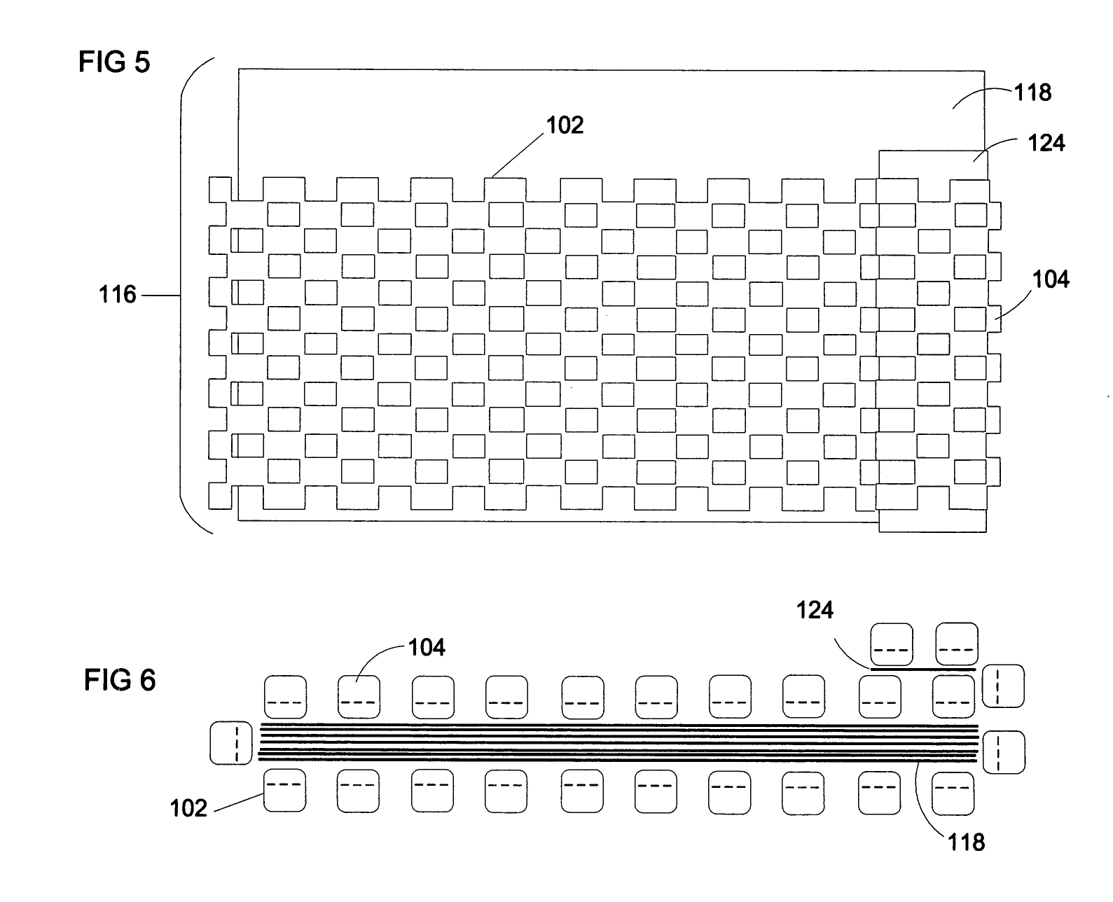

FIG. 5:
102. Padding
116. Unitary splint device
118. Resin-impregnated core
124. Double sided adhesive tape

FIG. 6:
102. Padding
116. Unitary splint device
118. Resin-impregnated core
124. Double sided adhesive tape

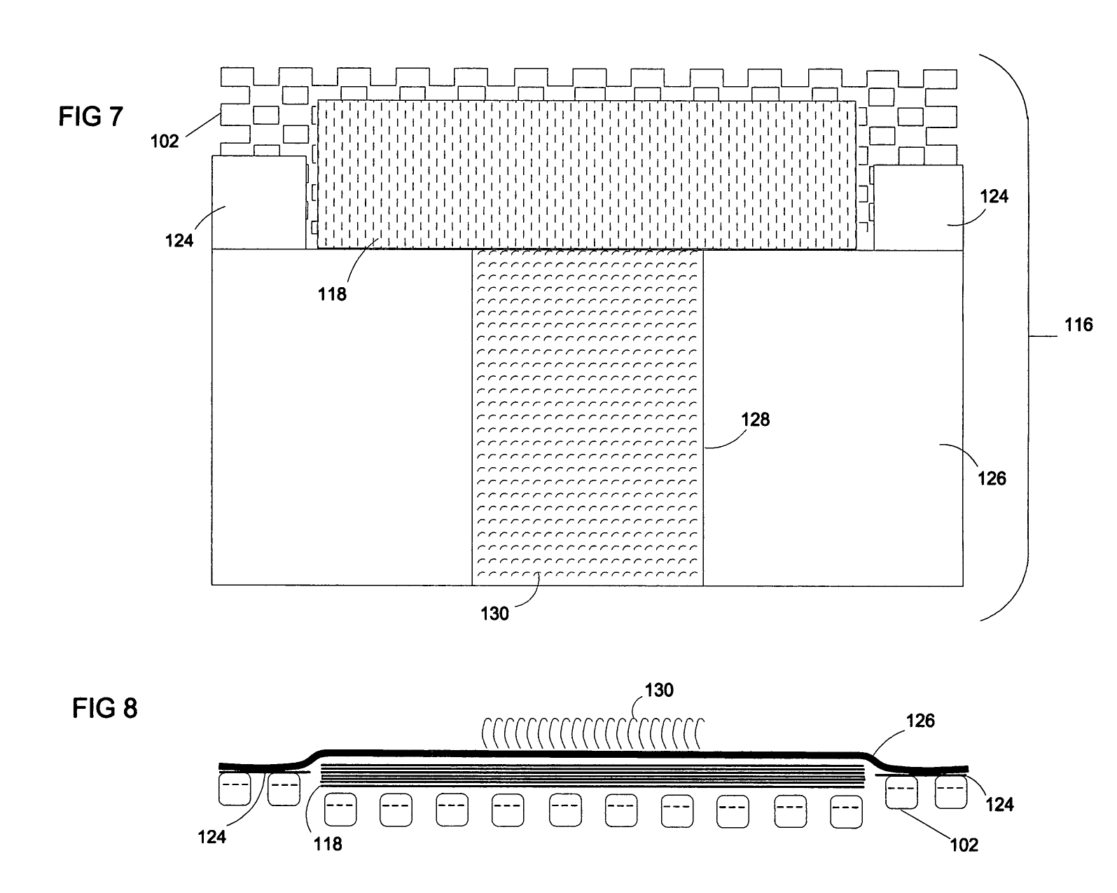

FIG. 7:
102. Padding
116. Unitary splint device
118. Resin-impregnated core
124. Double sided adhesive tape
126. Polyvinylchloride layer
128. Strip of hook-and-pile material
130. Male hook portion

FIG. 8:
102. Padding
116. Unitary splint device
118. Resin-impregnated core
124. Double sided adhesive tape
126. Polyvinylchloride layer
128. Strip of hook-and-pile material
130. Male hook portion

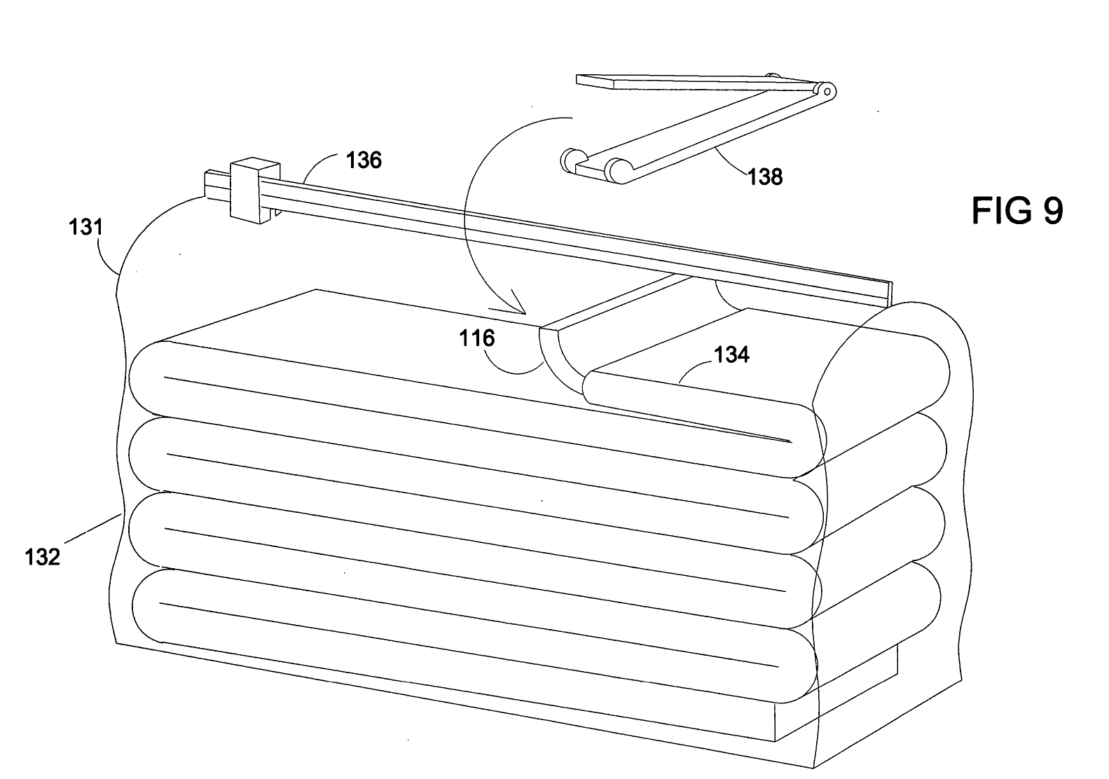

FIG. 9:
116. Unitary splint device
131 Air-tight pouch
132. Splint containment system
134. Sheath
136. Seal
138. Clamp

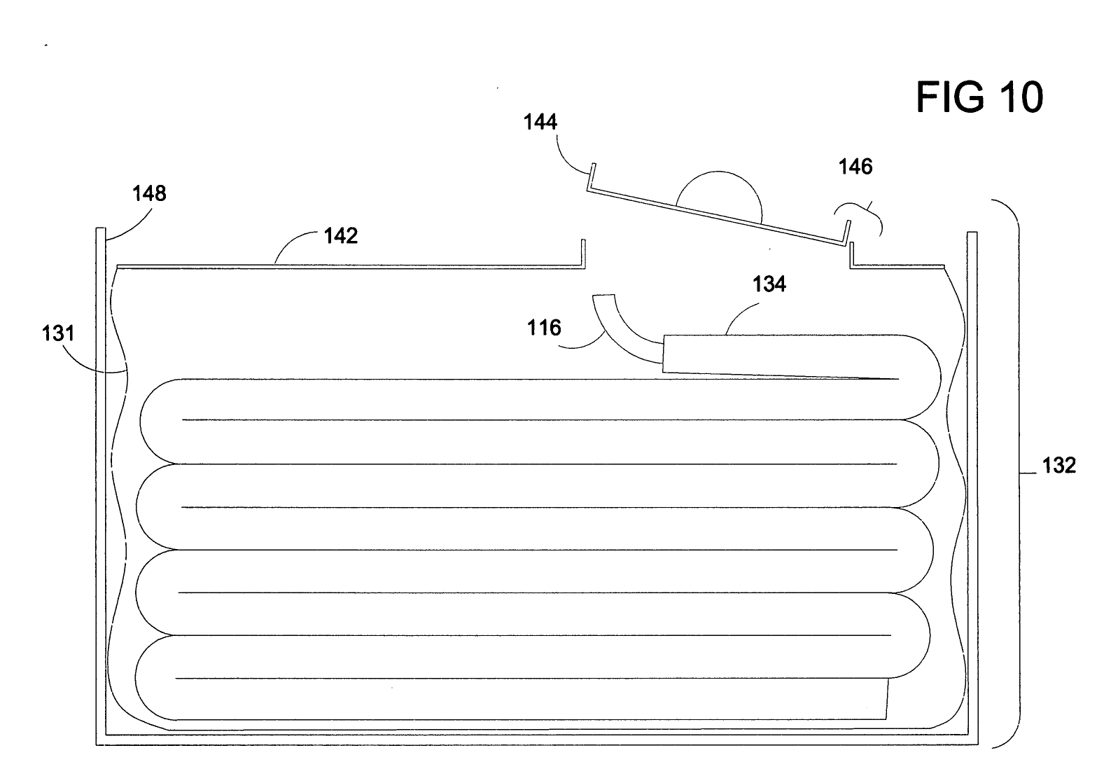

FIG. 10:
102. Padding
118. Resin-impregnated core
131 Air-tight pouch

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

FIG. 1 shows a front view of the padding used in the splint. Padding 102 is comprised of warp 110 and weft 112, which is a polyester material. Between-warp 110 and weft 112, are areas or patches of dense weave 114. This polyester scrim 108 is dipped in a bath of a hydrophilic material such as polyvinylchloride. Thus, pillows of polyvinylchloride adhere to the areas of the dense weave 114. But the open areas between the dense weaves do not hold any polyvinyl chloride. The result is that interspersed between pillows 104 are open spaces 106. These open spaces allow for the penetration of water, moisture, and air.

FIG. 2 is a cross section of the padding 102 seen in FIG. 1. Dense weave 114 of scrim 108 can be seen encased in pillows 104 of the PVC material. Between these pillows 104, are interspersed open spaces 106 as mentioned above.

FIG. 3 shows the front view of a preferred embodiment of the unitary splint device 116. A resin-impregnated core 118 is made up of multiple layers of resin-impregnated material. The cutaway padding 102 is shown surrounding and encasing the resin-impregnated core 118 within.

FIG. 4 shows a cross section view of the unitary splint device. Padding 102 encases this core 118 and after the padding is folded over to contain the core 118, the padding edges are bonded to each other with heat seal 122. This creates a unitary splint device.

FIGS. 5 and 6 depict an alternative embodiment of the unitary splint device 116. Again, on the front view is seen the padding 102 encasing the resin impregnated core 118. A strip of double-sided adhesive tape 124 is seen along one side. The cross section view shows that the padding 102 is wrapped around core 118 and that the double sided adhesive tape 124 is laid on one edge on the top the padding. When the padding is folded over, each of the opposing edges of the padding adheres to the adhesive material forming a unitary splint device.

One may note in FIG. 4 and FIG. 6 how easily water needed to activate the resin material can pass through the padding 102 to activate the resin material 118. One may also note how easily air and moisture can pass out of the splint material to maintain a dry contact area with the patient.

FIGS. 7 and 8 are cross-section views, respectively, of a second alternative embodiment of splint device 116. This drawing shows the splint from the outside with the padding 102 underneath. Padding 102 is applied against the patient's skin. The resin containing inner core 118 is placed on top of the padding 102 and on top of this are the polyvinylchloride water impermeable and moisture permeable layer 126. Over this is a strip 128 of a hook-and-pile/fabric hook material, such as Velcro™ containing the hook portion 130. This strip helps to prevent undesired migration of the overlying elastic bandage that holds the splint in place.

Double-sided adhesive strips 124 on each side connect the padding 102 to the polyvinylchloride layer 126, thereby forming a unitary device.

FIG. 9 shows a splint containment and dispenser system 132 comprised of sheath 134 that contains a splint 116, folded upon itself within a collapsible bag having a Ziploc™ type of seal 136 across the top. An alternative embodiment might include a method to fold the sheath 134 over on itself and then use a clip to provide an airtight seal. When a desired length of the splint material is pulled out of its container 132, it is cut and the end of splint 116 is placed back into container 132 and then the open end is resealed with clamp 138.

The sheath in this example is made from an aluminized thick plastic type material to prevent air and light from entering. After a desired length of splint is removed and cut off, the remainder is tucked back into its sheath and then back into the pouch, excess air is expelled, and the pouch is resealed. In this way, the pouch and the sheath form a double layer of protection for the, still soft, splinting material, preventing contact with air and humidity from outside its container/dispenser.

FIG. 10 illustrates a splint containment and dispenser system 132 comprising a sheath 134 containing the unitary splint device material 116, a rigid opaque outer shell 148, an air-tight collapsible pouch 131, a rigid opaque pouch top surface 142, and a pouch lid 144 having an openable and resealable air-tight seal 146.

To dispense splint material 116, the lid 144 is removed to allow the splint device material 116 to be extracted. Once the desired quantity is dispensed, it is cut free and the material remaining is pushed back into the, now shortened, sheath 134 and the airtight pouch 131. Excess air is expelled by pressing down on the rigid top 142 and the resealable lid 144 is then fitted back into its airtight seal 146.

The splint of fiberglass material in the form of heat-softened or curable organic polymer resins, coated on glass fibers formed into fabrics, and surrounded by the herein taught padding material, is wetted, then fitted to the body member and thereafter allowed to harden.

The splint and supported member, thus fitted, are, then, usually wrapped in an elastic bandage to hold the splint in place.

Such a splint is tolerant of and unaffected by water, as is the padding that surrounds it. It drains easily, dries quickly, and allows moisture to be wicked away from the skin. It is not weakened by dampness, nor does it move nor migrate nor tend to clump or bunch up when wetted, as cotton or other conventional fabrics do. By these qualities, it discourages maceration under the splint, thereby discouraging attack by bacteria, skin erosion and subsequently formed pustules, otherwise notable sources of injury and discomfort. Furthermore, it greatly alleviates potential for unpleasant odors that otherwise may develop due to retention of body oils and perspiration in the padding by allowing immersion of the splinted member without damage to the splint or padding.

The herein taught art additionally, in conditions of immersion, is superior to the previously taught materials having qualities of being water-impermeable while water-vapor-permeable. These qualities become moot because liquid water will inevitably enter the interior of the splint and liner by bypassing the edges of the protective materials. The problem to be addressed then becomes one of quickly getting the skin dry again. The herein taught art overcomes this problem, creating an advantage in that, without the ability to efficiently dry the previously taught padding under splints, immersing or otherwise wetting them becomes impractical or unfeasible. Thus, the instant technology makes immersion of the splinted limb no longer impractical.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Padded orthopaedic splint device comprising:
a splint core of flexible fabric that may be cured to hardness; and
a sleeve surrounding the splint core comprised at least partially of a scrim in net-like or web-like configuration coated with polyvinylchloride.

2. A device as in claim 1 wherein the scrim comprises polyester.

3. A device as in claim 1 also comprising a container from which said splint device may be dispensed, said container being so disposed and configured as to be repeatedly openable and resealable to an essentially hermetic seal.

4. A device as in claim 1 also comprising a container that is light, air, and, or, or humidity resistant.

5. A device as in claim 1 also comprising a container from which said splint device may be dispensed, said container being so disposed and configured as to be repeatedly openable and resealable to an essentially hermetic seal, and comprising one or more compressible walls, sides or faces, so configured that excess air may be expelled from inside by compressing the container via pressure on one or more of its surfaces.

6. A device as in claim 3 wherein the said container is configured with an air-tight lid.

7. A device as in claim 3 wherein the said container is configured with an air tight ZIPLOCK™ type seal.

8. A device as in claim 3 wherein a sheath is disposed about the splint device inside the said container, so configured as to allow a quantity of the splint device to be uncovered or or unwrapped and drawn from the said sheath, and the sheath resealed.

9. A device as in claim 3 wherein a sheath is disposed about the splint device inside the said container, such that the sheath may provide an additional layer of essentially hermetic protection and thereby further inhibit air and humidity circulation in the container from coming in contact with the splint.

10. A device as in claim 3 wherein a sheath is disposed about the splint device inside the said container, such that the sheath may limit light exposure of the splint device.

11. A re-usable splint padding of scrim in net-like or web-like configuration and coated with polyvinylchloride.

12. A device as in claim 11 wherein the scrim comprises polyester.

13. A device as in claim 11 wherein the reusable padding is wrapped around the splint material and secured by heat sealing.

14. A device as in claim 11 wherein the reusable padding is wrapped around the splint material and secured by adhesive.

15. A device as in claim 11 also comprising polyester Velcro™ fabric-hook type material so disposed as to inhibit or limit movement or slippage of such bandage as would be wrapped around the splint and a splinted limb to secure the splint in place.
